(19) United States
(12) Patent Application Publication
(10) Pub. No.: US 2011/0021567 A1
(43) Pub. Date: Jan. 27, 2011

(54) PREPARATION OF LENALIDOMIDE

(75) Inventors: Surya Narayana Devarakonda, Hyderabad (IN); Sesha Reddy Yarraguntla, Hyderabad (IN); Vamsi Krishna Mudapaka, Khammam (IN); Rajasekhar Kadaboina, Hyderabad (IN); Veerender Murki, Hyderabad (IN); Amarendhar Manda, Hyderabad (IN); Venkata Rao Badisa, Krishna (IN); Naresh Vemula, Nalgonda (IN); Rama Seshagiri Rao Pulla, Hyderabad (IN); Venu Nalivela, Hyderabad (IN)

Correspondence Address: DR. REDDY''S LABORATORIES, INC., 200 SOMERSET CORPORATE BLVD, SEVENTH FLOOR, BRIDGEWATER, NJ, 08807-2862, US

(73) Assignees: DR. REDDY'S LABORATORIES LTD., Hyderabad 500 016, Andhra Pradesh (IN); DR. REDDY'S LABORATORIES, INC., Bridgewater, NJ (US)

(21) Appl. No.: 12/921,613

(22) PCT Filed: Mar. 11, 2009
(86) PCT No.: PCT/US09/36773

§ 371 Date: Sep. 9, 2010

Related U.S. Application Data
(60) Provisional application No. 61/053,710, filed on May 16, 2008.
(60) Provisional application No. 61/100,106, filed on Sep. 25, 2008.
(60) Provisional application No. 61/143,966, filed on Jan. 12, 2009.

(30) Foreign Application Priority Data
Mar. 11, 2008 (IN) 605/CHE/2008
Jun. 11, 2008 (IN) 1422/CHE/2008
Nov. 19, 2008 (IN) 2866/CHE/2008

(51) Int. Cl. C07D 401/04; A61K 31/454; A61P 35/00

(52) U.S. Cl. 514/323; 546/200

(57) ABSTRACT

Processes for the preparation of substantially pure lenalidomide. The application also relates to an enriched, substantially pure, and pure amorphous form of lenalidomide and solid dispersions containing amorphous lenalidomide.

### INTRODUCTION

In an aspect, the present application relates to processes for the preparation of substantially pure lenalidomide, free from its impurities.

An aspect of the present application also relates to an enriched, substantially pure, or pure amorphous form of lenalidomide, and to solid dispersions containing amorphous lenalidomide.

The drug compound having the adopted name “lenalidomide” has a chemical name 3-(4-amino-1-oxo-1,3-dihydro-2H-isoindol-2-yl)piperidine-2,6-dione, and is structurally represented by Formula I.

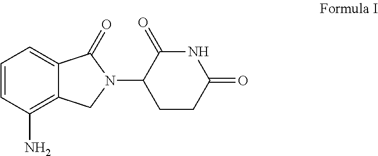

Lenalidomide, a thalidomide analogue, was initially intended for use as a treatment for multiple myeloma, for which thalidomide is an accepted therapeutic modality, but has also shown efficacy in the hematological disorders known as the myelodysplastic syndromes. The exact mechanism of the immuno-modulatory drugs (e.g., thalidomide, CC-4047/actimid and lenalidomide) is not known. Apart from interfering with the immune system, they are also found to be active for angiogenesis. With myelodysplastic syndromes, the encouraging results of lenalidomide were also obtained in patients with deletion 5q cytogenetic abnormality.

Lenalidomide was approved by the U.S. Food and Drug Administration on Dec. 27, 2005 for treating patients with low or intermediate-1 risk MDS with 5q—with or without additional cytogenetic abnormalities.

The drug is commercially marketed in products sold by Celgene Corporation under the brand name REVLIMID™ in the form of capsules having the strengths 5 mg, 10 mg, 15 mg, and 25 mg.

Muller et al., in U.S. Pat. No. 5,635,517 disclose substituted 1-oxo-2-(2,6-dioxopiperidin-3-yl) isoindolines derivatives, pharmaceutical compositions containing these compounds and their use in the treatment of cancer. It also discloses a process for the preparation of these compounds, which involves hydrogenation of a nitro group to an amino group, using palladium on carbon in 1,4-dioxane solvent.

Muller et al., in U.S. Patent Application Publication No. 2006/0052609, disclose another process for the preparation of lenalidomide. The process involves the hydrogenation of (S)- or racemic 3-(4-nitro-1-oxo-1,3-dihydroisoindol-2-yl)-piperidine-2,6-dione using 10% palladium on carbon in methanol, to form (S)- or racemic 3-(4-amino-1-oxo-1,3-dihydro-2H-isoindol-2-yl)piperidine-2,6-dione.

Palle et al., in Indian Application No. 047/CHE/2006, published on Nov. 23, 2007, disclose a process for the preparation of lenalidomide comprising hydrogenating 3-(4-nitro-1-oxo-1,3-dihydroisoindol-2-yl)-piperidine-2,6-dione using 10% palladium on carbon in a mixture of solvents comprising methanol and N,N-dimethylformamide.

The above process conditions may lead to the presence of impurities like unreacted starting materials and derivatives of lenalidomide, in the final product. The presence of these impurities may depend on the solvent, reaction conditions and the like in the hydrogenation reaction. None of the above noted documents mentions suitable conditions to avoid the formation of impurities, or purification techniques to reduce those impurities.

Hence, there is a need in the art for improved processes that produce lenalidomide in improved purity and yield, and are suitable for use on an industrial scale.

Chen et al., in International Application Publication No. WO 2005/023192, disclose polymorphic forms of lenalidomide, designated as forms A, B, C, D, E, F, G, and H. Further, the publication also discloses pharmaceutical compositions comprising the various crystalline forms of lenalidomide and mixtures of crystalline forms having greater than 50% crystallinity.

A single compound may give rise to a variety of solid forms having distinct physical properties. The variation in the physical properties frequently results in differences in bioavailability, stability, etc.

Some polymorphic forms of drug substances suffer from the drawbacks of spontaneous conversion to other crystalline forms during storage, resulting in concomitant change, not only in the physical form and shape of the drug crystals, but also associated changes in distinct physical properties. Generally, the forms will revert to a more thermodynamically stable form, often a form with lower solubility. Such a thermodynamically stable form may sometimes result in a reduced or suboptimal bioavailability, especially for oral administration.

There remains a continuing need, not only for a pure or substantially pure amorphous form of lenalidomide or its solid dispersions that are stable, but also for processes to produce lenalidomide, which are amenable to scale-up for commercial production quantities and yield both formulation and therapeutic benefits.

### SUMMARY

In an aspect, the present invention provides improved processes for the preparation of substantially pure lenalidomide, substantially free from its impurities.

An aspect of the present invention provides an amorphous form of lenalidomide, and solid dispersions comprising amorphous lenalidomide and a pharmaceutically acceptable carrier.

In an aspect of the present invention, there are provided processes for the preparation of substantially pure lenalidomide, an embodiment comprising one or more of:

i) reacting methyl 2-halomethyl-3-nitrobenzoate of Formula III, where X is a halogen,

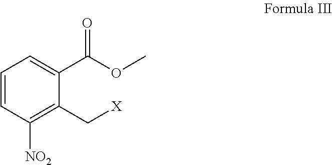

with α-aminoglutarimide hydrochloride of Formula IV,

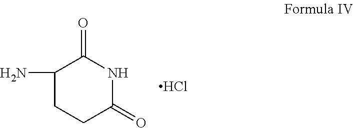

using triethylamine in the presence of a solvent such as N-methylpyrrolidone or acetonitrile, to afford 3-(4-nitro-1-oxo-1,3-dihydroisoindol-2-yl)piperidine-2,6-dione of Formula II; and

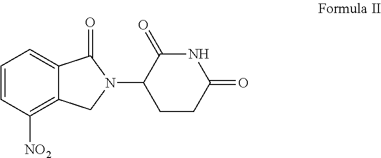

ii) hydrogenating 3-(4-nitro-1-oxo-1,3 dihydroisoindol-2-yl)-piperidine-2,6-dione of Formula II using a hydrogenation catalyst in a solvent and in the presence of an acid, to provide lenalidomide of Formula I.

In another aspect, the present invention provides an acid addition salt of lenalidomide, which may be utilized as an intermediate in the preparation of substantially pure lenalidomide. In an embodiment, the present invention provides an alkyl- or aryl-sulfonate salt of lenalidomide, such as a methanesulfonate salt of lenalidomide.

In another aspect, there is provided substantially pure lenalidomide having a purity greater than about 99% by weight, as determined using high performance liquid chromatography (HPLC).

In another aspect, the present invention provides an amorphous form of lenalidomide.

In yet another aspect, the present invention provides a solid dispersion comprising amorphous lenalidomide and a pharmaceutically acceptable carrier.

In an aspect, there are provided processes for preparing amorphous lenalidomide, an embodiment comprising at least one of:

a) providing a solution of lenalidomide in a solvent or a mixture of solvents,

b) removing the solvent from the solution of a); and

c) optionally, drying a solid formed in b) to afford the desired amorphous form of lenalidomide.

In yet another aspect there are provided processes for preparing a solid dispersion comprising amorphous lenalidomide, an embodiment comprising at least one of:

a) providing a solution containing lenalidomide and a pharmaceutically acceptable carrier in a solvent or a mixture of solvents;

b) removing the solvent from the solution of a); and

c) optionally, drying a solid formed in b) to afford the desired amorphous dispersion of lenalidomide.

In a further embodiment, there are provided processes for preparing amorphous lenalidomide, an embodiment comprising milling a lenalidomide crystalline material to afford the amorphous form of lenalidomide.

Lenalidomide in amorphous form of the present application is sufficiently stable and well suited for use in pharmaceutical formulations, which are useful in the treatment of disease, including, but not limited to, multiple myeloma.

### BRIEF DESCRIPTION OF THE DRAWINGS

FIG. 1 is an X-ray powder diffraction (XRD) pattern of a methanesulfonate salt of lenalidomide, prepared according to Example 2.

FIG. 2 is a differential scanning calorimetry (DSC) curve of a methanesulfonate salt of lenalidomide, prepared according to Example 2.

FIG. 3 is a thermogravimetric analysis (TGA) curve of a methanesulfonate salt of lenalidomide, prepared according to Example 2.

FIG. 4 is an XRD pattern of amorphous lenalidomide, prepared according to Example 8.

FIG. 5 is an XRD pattern of lenalidomide prepared according to Example 9.

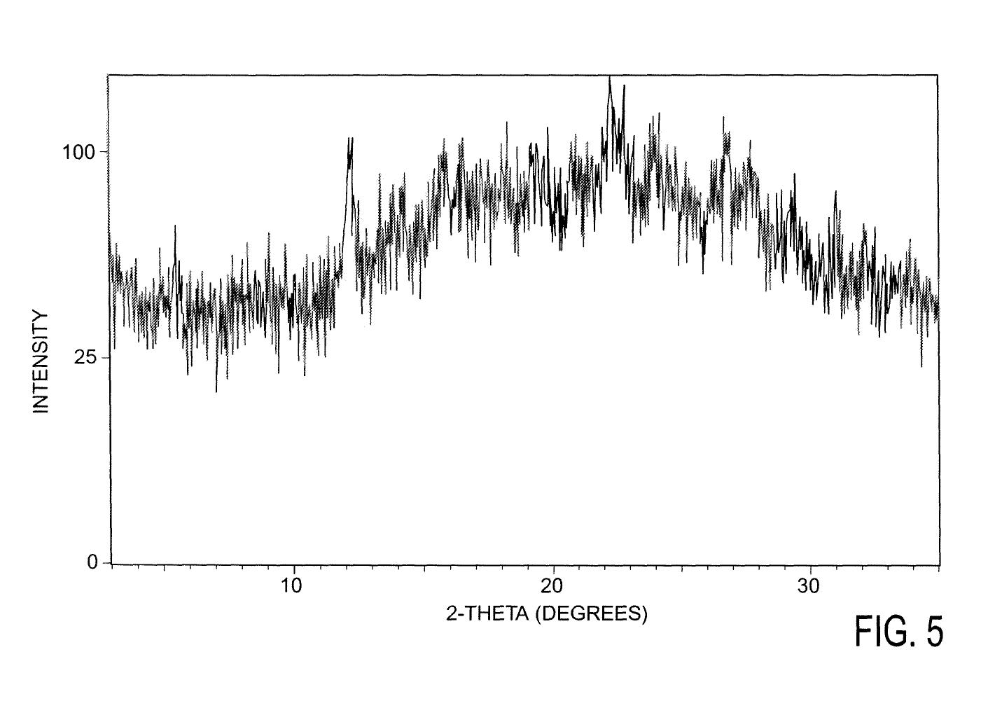

FIG. 6 is an XRD pattern of a solid dispersion of lenalidomide, prepared according to Example 10.

FIG. 7 is an XRD pattern of lenalidomide, prepared according to Example 3.

FIG. 8 is an XRD pattern of lenalidomide, prepared according to Example 4.

### DETAILED DESCRIPTION

An aspect of the present invention provides improved processes for the preparation of substantially pure lenalidomide, free from its impurities.

Aspects of the present application also provide an amorphous form of lenalidomide and a solid dispersion comprising amorphous lenalidomide and a pharmaceutically acceptable carrier.

In an embodiment of the present invention, a process for the preparation of substantially pure lenalidomide comprises one or more of:

i) reacting methyl 2-halomethyl-3-nitrobenzoate of Formula III,

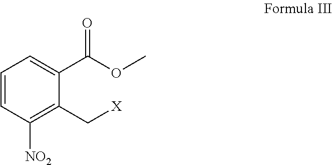

where X is a halogen, with α-aminoglutarimide hydrochloride of Formula IV,

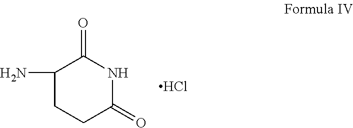

using a base in the presence of a N-methylpyrrolidone or acetonitrile solvent to afford 3-(4-nitro-1-oxo-1,3-dihydroisoindol-2-yl)-piperidine-2,6-dione of Formula II; and

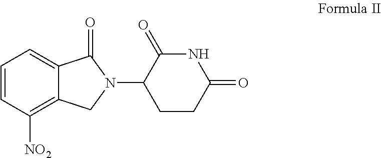

ii) hydrogenating 3-(4-nitro-1-oxo-1,3-dihydroisoindol-2-yl)-piperidine-2,6-dione of Formula II using a hydrogenation catalyst in a solvent and in the presence of an acid, to provide lenalidomide of Formula I.

The steps for this process are separately described below.

Step (i) involves reacting methyl 2-halomethyl-3-nitrobenzoate of Formula III with α-aminoglutarimide hydrochloride of Formula IV using triethylamine in presence of a N-methylpyrrolidone (NMP) or acetonitrile solvent, to afford 3-(4-nitro-1-oxo-1,3-dihydroisoindol-2-yl)-piperidine-2,6-dione of Formula II.

The compound of Formula III may be obtained by methods known in the art.

Solvents that may be used for the preparation of Formula II may also include nitriles such as, for example, propionitrile, and the like.

The quantity of solvent used for the preparation of compound of Formula II may range from about 5 mL to about 10 mL, per gram of the compound of Formula III.

In an embodiment, the quantity of the solvent used is about 10 volumes with respect to the weight of Formula III to provide the compound of Formula II with high purity and yield.

The amount of base, for example, triethylamine, used for preparing the compound of Formula II may range from about 1 to about 3 or more molar equivalents, per molar equivalent of Formula III.

The addition of the base to the reaction mass may be carried out in a single or multiple portions. The base may be added in equal portions or the size of the portions may be different. The entire quantity of the base may be added in about 2 to about 5 or more portions. The time between additions of the portions may vary, such as from about 30 minutes to about 3 hours, or more.

The reaction of step (i) may be carried out at temperatures of about 20° C. to about 160° C., or about 25° C. to about 60° C.

The compound of Formula II may be isolated by the techniques known in the art.

The compound, 3-(4-nitro-1-oxo-1,3-dihydroisoindol-2-yl)-piperidine-2,6-dione of Formula II obtained by the process of the present invention can have a purity of more than about 99.5%, or about 99.7%, by weight as determined using high performance liquid chromatography (HPLC).

Step ii) involves hydrogenating 3-(4-nitro-1-oxo-1,3-dihydroisoindol-2-yl)-piperidine-2,6-dione of Formula II, using a hydrogenation catalyst in a solvent and in the presence of an acid, to provide lenalidomide of Formula I.

The hydrogenation reaction is conducted using various catalysts, including but without limitation thereto: metal catalysts such as palladium, platinum, nickel, iridium, ruthenium, and the like on a carbon or other support; a transition metal catalyst in combination with an acid such as iron/HCl, Zn/HCl, Sn/HCl, Zn/acetic acid, or Zn/ammonium formate; Raney nickel; and the like. A catalyst may be a chemical reducing agent such as stannous chloride (SnCl₂), ferric chloride (FeCl₃), or zinc, in the presence of an acid like acetic acid or hydrochloric acid, or a base like hydrazine. A useful catalyst is palladium on carbon.

The concentrations of palladium on the support, such as carbon, that can be used for the hydrogenation reaction may range from about 1% to about 30%, or about 5% to 10%, or about 10% by weight.

For example, a quantity of 10% Pd on carbon that is used in the reaction of step (ii) may range from about 0.05 to 0.15 grams, per gram of 3-(4-nitro-1-oxo-1,3-dihydroisoindol-2-yl)-piperidine-2,6-dione of Formula II.

The solvents that may be used in the hydrogenation reaction include, but are not limited to: water; alcohols like methanol, ethanol, n-propanol, isopropyl alcohol, n-butanol, and the like; ketonic solvents like acetone, ethyl methyl ketone, methyl isobutyl ketone and the like; N,N-dimethylformamide (DMF); N,N-dimethylacetamide; dimethylsulfoxide (DMSO); and mixtures thereof. For example, water or methanol may be used as the solvent in the hydrogenation reaction.

The quantity of solvent used for the hydrogenation reaction is less than about 50 times the weight of the compound of Formula II and may also depend on the solvent selected.

Acid that may be used in the hydrogenation reaction include inorganic acids and organic acids, such as but not limited to: organic acids like alkyl- and aryl-sulfonic acids, such as methanesulfonic acid, formic acid, acetic acid, trifluoroacetic acid, or their salts; and inorganic acids such as hydrochloric acid, sulfuric acid, phosphoric acid, and the like.

The use of an acid in the hydrogenation reaction of the present invention reduces amounts of the organic solvent and also reduces the duration of reaction time, and provides better yield and purity of lenalidomide, thereby making the process more reproducible and suitable for industrial scale use.

The reaction of step (ii) may be carried out at temperatures ranging from about 20° C. to about 60° C., or about 25° C. to about 35° C.

The reaction mixture of step (ii) contains an acid addition salt of lenalidomide. In an embodiment, the salt is an alkyl- or aryl-sulfonate salt of lenalidomide, such as a methanesulfonate salt of lenalidomide.

In an embodiment, the reaction of step ii) may be carried out by hydrogenating 3-(4-nitro-1-oxo-1,3-dihydroisoindol-2-yl)-piperidine-2,6-dione of Formula II using 10% Pd/C in the presence of water or methanol as the solvent and methanesulphonic acid, to provide a methanesulfonate salt of lenalidomide.

The acid addition salt of lenalidomide obtained from step (ii) of the above reaction may optionally be isolated or in situ converted to lenalidomide.

In an embodiment, the acid addition of salt of lenalidomide is isolated after reduction of the 3-(4-nitro-1-oxo-1,3-dihydroisoindol-2-yl)-piperidine-2,6-dione of Formula II from the reaction mixture of step (ii).

For example, the acid addition of salt of lenalidomide may be isolated by filtering the reaction mixture of step (ii) and optionally concentrating to an extent where the precipitation of solid may begin from the solution. Generally, the concentration may be terminated when the quantity of solvent becomes less than about 15 volumes with respect to the weight of 3-(4-nitro-1-oxo-1,3-dihydro-isoindol-2-yl)-piperidine-2,6-dione of Formula II. The suspension obtained may be maintained further at temperatures lower than the concentration temperatures such as, for example, below about 40° C., for a period of time as required for the desired extent of isolation of an acid addition salt of lenalidomide.

The exact cooling temperature and time required for crystallization may be readily determined by a person skilled in the art and will also depend on parameters such as concentration and temperature of the solution or slurry.

The obtained acid addition salt of lenalidomide may be optionally be further purified using suitable purification techniques such as recrystallization, slurrying in a solvent or mixture of solvents, using a solvent and anti-solvent crystallization technique, and the like.

In an embodiment, the acid addition salt obtained from the present invention is a methanesulfonate salt of lenalidomide and may be characterized by any one or more of its X-ray powder diffraction (XRD) pattern, differential scanning calorimetry (DSC) curve, thermogravimetric analysis (TGA) curve, and infrared absorption spectrum.

A methanesulfonate salt of lenalidomide obtained by the process of the present invention may be characterized by any one or more of:

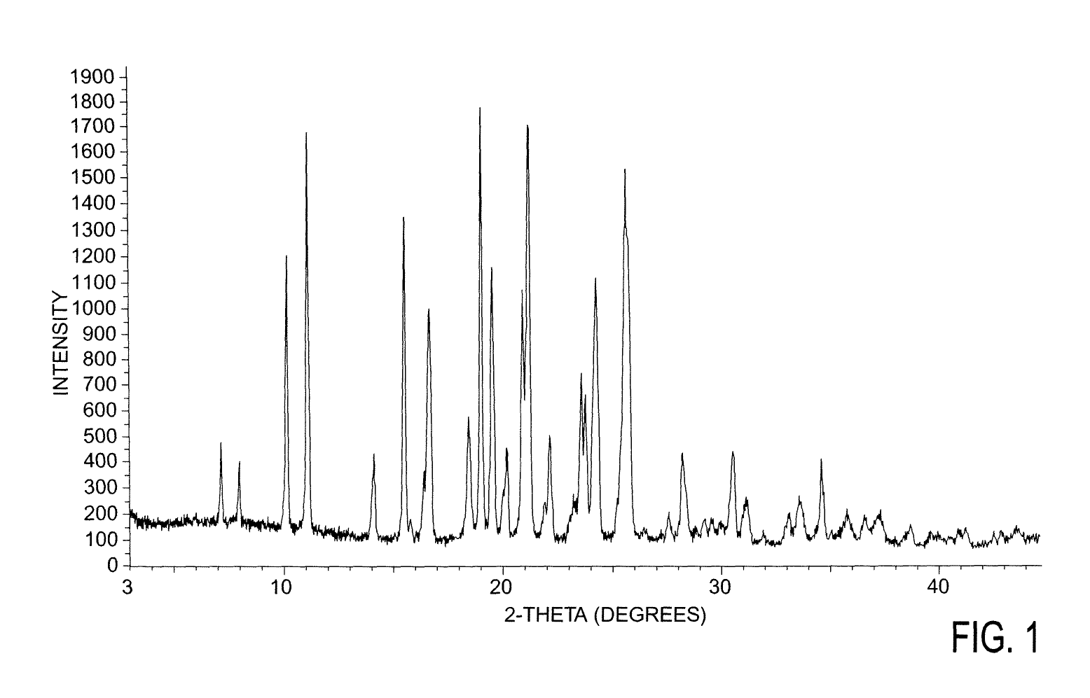

(a) An XRD pattern substantially in accordance with FIG. 1.

(b) An XRD pattern having characteristic 2-theta peaks at about 10.1, 11.0, 15.5, 16.6, 16.7, 18.5, 19.1, 19.5, 20.3, 21.0, 21.1, 22.1, 23.6, 23.8, 24.2, 25.6, 25.8, and 30.6, ±0.2 degrees.

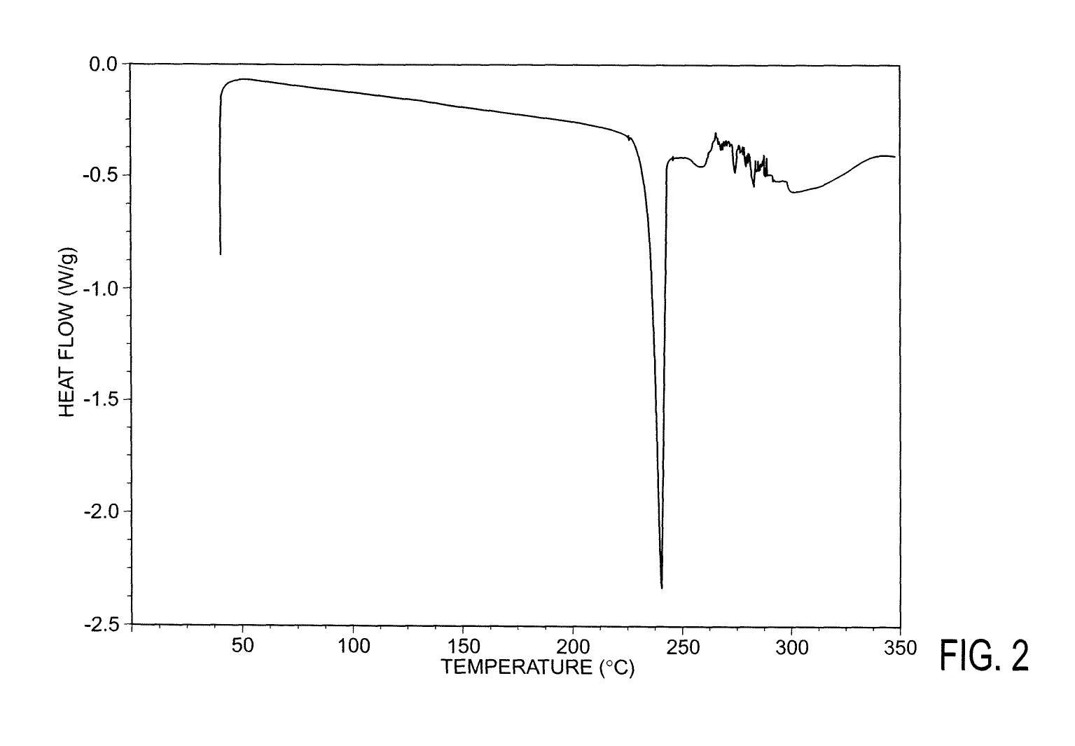

(c) A DSC curve substantially in accordance with FIG. 2.

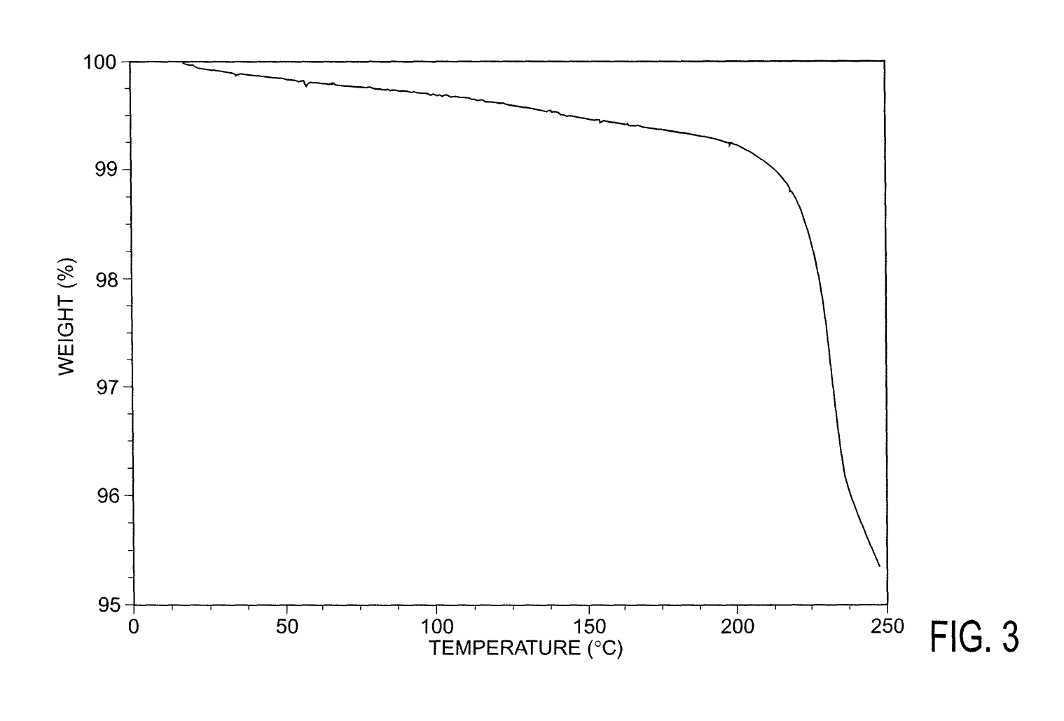

(d) A thermogravimetric analysis curve substantially in accordance with FIG. 3.

In an embodiment, the present invention provides a process for the preparation of an acid addition salt of lenalidomide, comprising:

(i) providing a solution of lenalidomide and an acid in a solvent; and

(ii) isolating the acid addition salt.

The acid addition salt of lenalidomide, which is isolated, may be further converted into lenalidomide by reaction with a base in the presence of a solvent.

Suitable bases that may be used include but are not limited to: organic bases such as, for example, pyridine, imidazole, N-methylmorpholine, and alkyl amines such as triethylamine, methylamine, isopropylamine, diisopropylethylamine, and the like; and inorganic bases such as, for example, ammonia, sodium hydroxide, potassium hydroxide, lithium hydroxide, sodium carbonate, potassium carbonate, sodium bicarbonate, potassium bicarbonate, and the like.

The quantity of base, which is used in the present invention, may range from about 0.5 to about 2.5 molar equivalents, or 1 molar equivalent, per equivalent of acid addition salt of lenalidomide.

The above reaction may be carried out in solvents including, but not limited to: water; alcohols such as methanol, ethanol, n-propanol, isopropyl alcohol, n-butanol, and the like; ketonic solvents such as acetone, ethyl methyl ketone, methyl isobutyl ketone and the like; nitriles such as acetonitrile, propionitrile and the like; and mixtures thereof; optionally in combination with ethers such as methyl t-butyl ether (MTBE) and the like. For example, isopropanol, methanol, methanol/MTBE or acetonitrile/MTBE may be used.

The base selected is added to the obtained reaction solution at temperatures of about 20° C. to about 60° C. and maintained for a suitable period of time to provide lenalidomide.

In another embodiment the acid addition salt of lenalidomide obtained by the reduction of the 3-(4-nitro-1-oxo-1,3-dihydroisoindol-2-yl)piperidine-2,6-dione of Formula II in step (ii) may be converted in situ to lenalidomide.

For example, the reaction mixture of step (ii) comprising an acid addition of salt of lenalidomide may be treated with a suitable base in a solvent to form lenalidomide. Suitable bases that may be used include, but are not limited to: organic bases such as, for example, pyridine, imidazole, N-methylmorpholine and alkylamines such as triethylamine, methylamine, isopropylamine, diisopropylethylamine, and the like; and inorganic bases such as, for example, ammonia, sodium hydroxide, potassium hydroxide, lithium hydroxide, sodium carbonate, potassium carbonate, sodium bicarbonate, potassium bicarbonate and the like. The selected base may be used in the form of a solution, if desired.

For example aqueous sodium bicarbonate solution may be used as the base. The base selected is added to the obtained reaction solution at temperatures of about 20° C. to about 60° C., and maintained for a suitable period of time to form lenalidomide.

The polymorphic nature of the lenalidomide obtained from the acid addition of salt of lenalidomide may depend upon the solvent utilized.

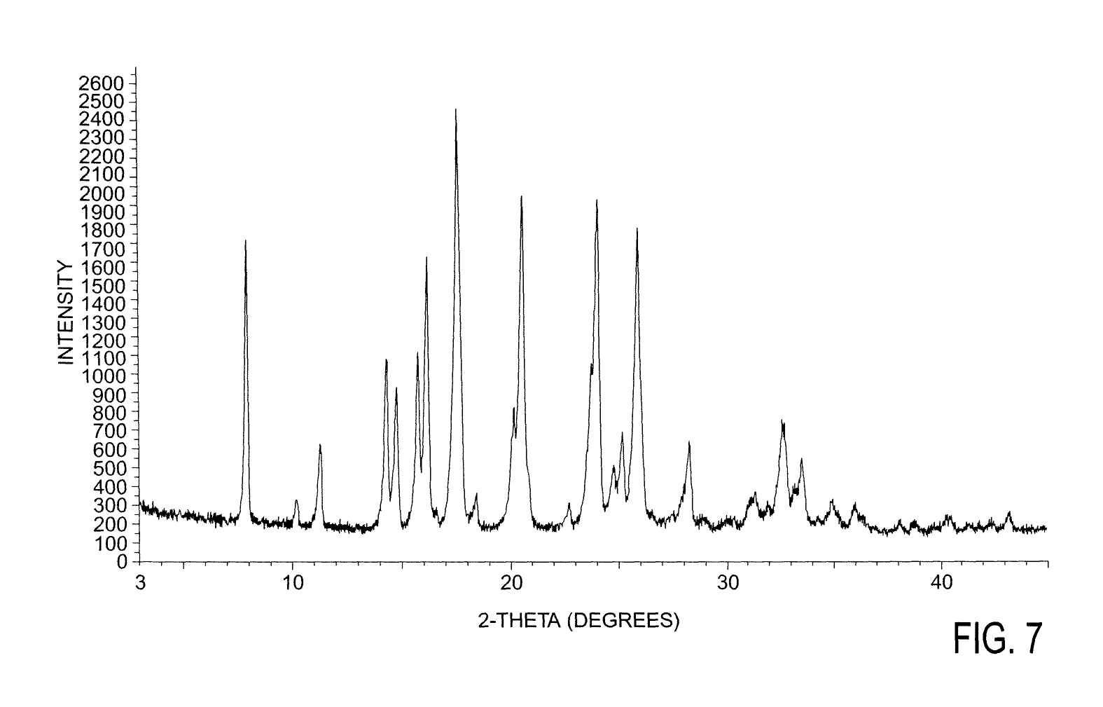

For example, lenalidomide obtained by the reaction of a methanesulfonic acid salt of lenalidomide with a base in the presence of an non-aqueous solvent has an XRD pattern substantially in accordance with FIG. 7.

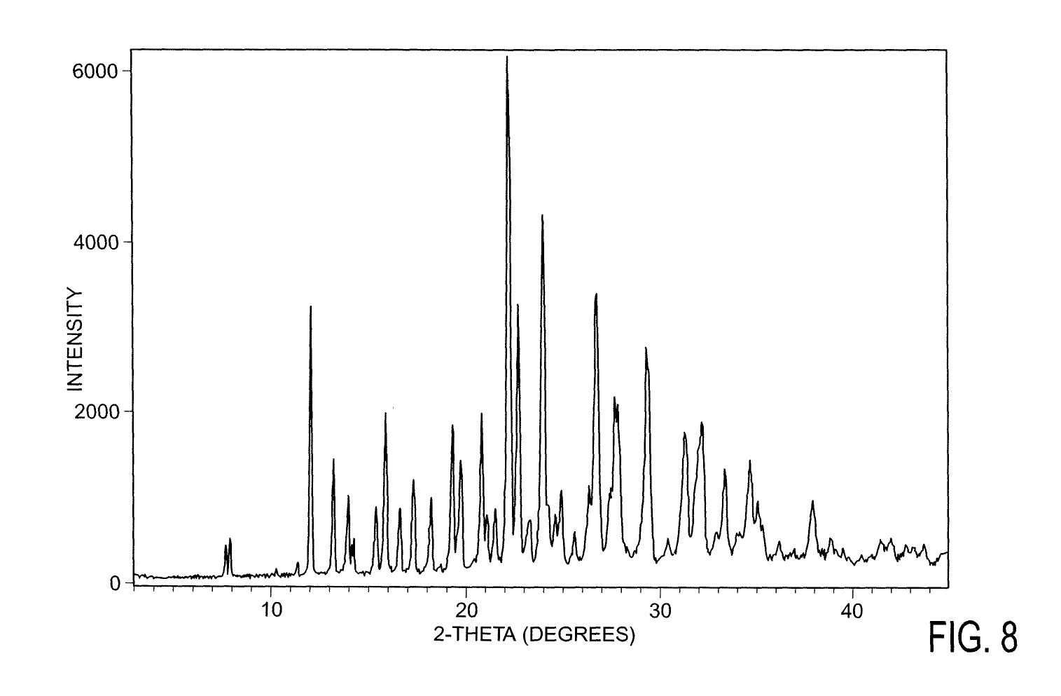

Further, lenalidomide obtained by the reaction of a methanesulfonic acid salt of lenalidomide with a base in the presence of an aqueous solvent has an XRD pattern substantially in accordance with FIG. 8.

An overall process embodiment of the present invention is represented in Scheme 1.

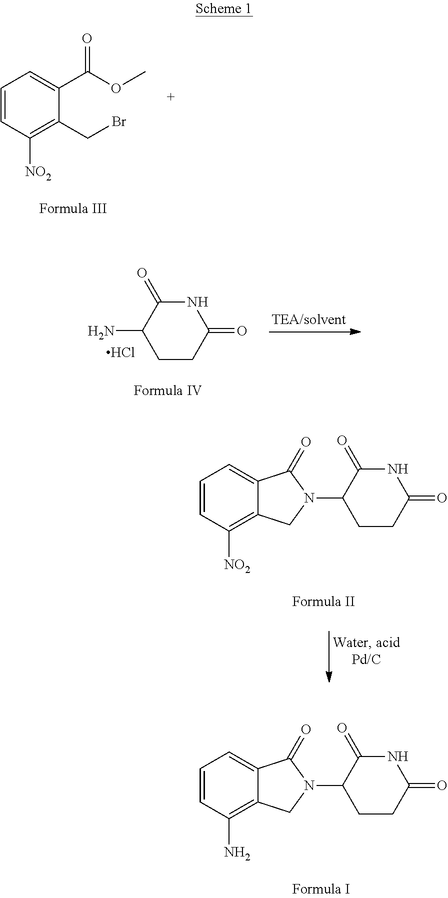

The above-described processes of the present invention provide substantially pure lenalidomide having a chemical purity more than about 99% by weight, such as determined using high performance liquid chromatography (HPLC).

In an embodiment, lenalidomide obtained by the processes of the present invention has a particle size distribution with D₉₀ less than about 500 μm, or less than about 200 μm.

The “D values” are useful ways for indicating a particle size distribution. D₉₀ refers to the value of particle size for which 90 percent of the particles have a size smaller than the value given. There are various methods for determining D₉₀ including laser light diffraction, such as using equipment from Malvern Instruments Ltd. (Malvern, Worcestershire, United Kingdom). There is no specific lower limits for any of the D values.

Lenalidomide obtained from the processes of the present application may be used for the preparation of amorphous lenalidomide and solid dispersions comprising lenalidomide.

The present application provides an amorphous form of lenalidomide, which may be characterized by its X-ray powder diffraction (XRD) pattern, as well as using thermal techniques such as differential scanning calorimetry (DSC) and thermogravimetric analysis (TGA).

In embodiments, the amorphous form of the present application may be in a pure amorphous form, however, in certain embodiments, there is provided an amorphous enriched form, wherein the amorphous content in the solid lenalidomide is about 60% or more by weight. It may be substantially pure amorphous form, which has about 90% by weight or more of the amorphous form. Also, it may be pure amorphous form, which has about 98% by weight or more of the amorphous form.

Amorphous and solid dispersions, unless stated otherwise, may be characterized by their X-ray powder diffraction patterns, differential scanning calorimetry curves, thermogravimetric analysis curves and infrared absorption spectra.

XRD data reported herein were obtained using a Bruker AXS D8 Advance powder X-ray diffractometer with copper Kα radiation, having the wavelength 1.5418 Å.

Differential scanning calorimetric analyses were carried out in a DSC Q200 V23 12 Build 103 instrument with a ramp of 5° C./minute, a modulation time of 60 seconds and a modulation temperature of ±1° C. The starting temperature was 0° C. and ending temperature was 350° C.

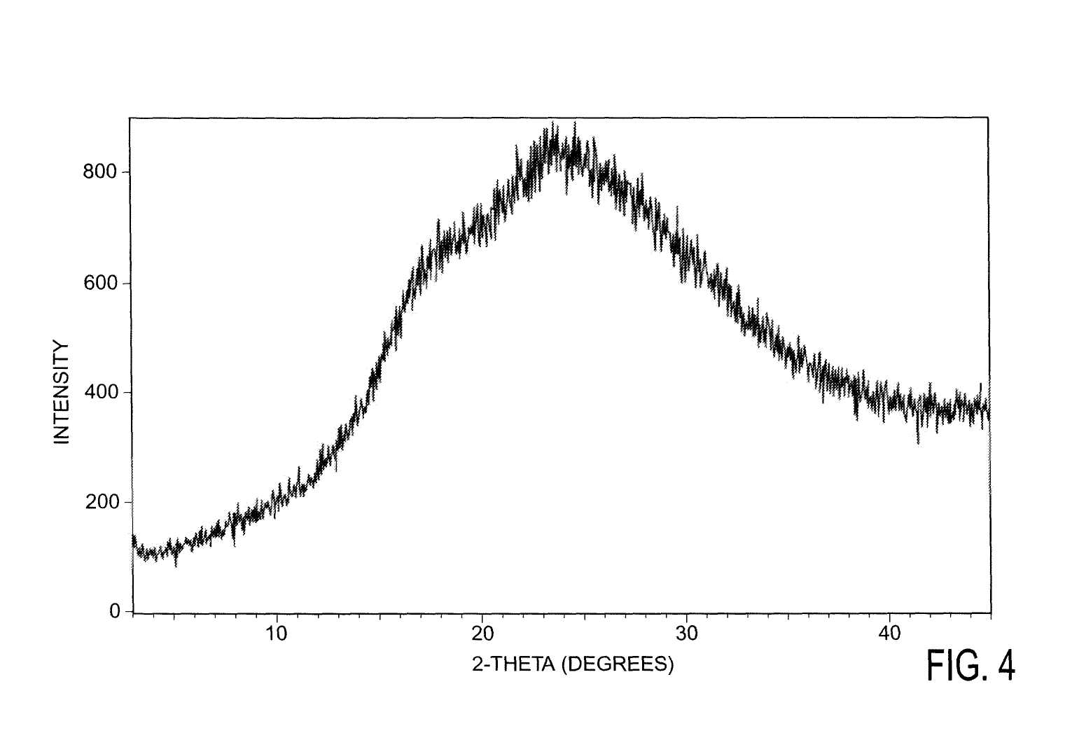

In one embodiment, there is provided an amorphous lenalidomide, characterized by its XRD pattern substantially in accordance with FIG. 4.

In an embodiment, there is provided a process for preparing amorphous lenalidomide, comprising removing solvent from a solution of lenalidomide.

A solution of lenalidomide may be provided by dissolving lenalidomide in a solvent or a mixture of solvents, or such a solution may be obtained directly from a reaction in which lenalidomide is formed. Any polymorphic form may be used in the preparation of solution, such as crystalline forms including solvates and hydrates.

Solvents which may be used for dissolving lenalidomide include, but are not limited to, water, organic solvents like C₁-C₄ alcohols, C₁-C₄ alkyl nitriles, C₃-C₅ alkyl amides, C₃-C₉ ketones, and mixtures thereof. Specific examples of solvents that may be utilized for the present invention include methanol, acetonitrile, N,N-dimethylformamide, N,N-dimethylacetamide, acetone, and mixtures thereof.

Typical dissolution temperatures can range from about 20° C. to about 100° C., depending on the solvent used for dissolution. Any other temperature is also acceptable as long as a clear solution of lenalidomide is provided.

The quantity of solvent used for dissolution depends on the solvent and the dissolution temperature adopted. The concentration of lenalidomide in the solution may generally range from about 0.1 to about 10 g/mL in the solvent.

Optionally, the solution obtained above may be filtered to remove any undissolved particles, prior to further processing. The undissolved particles may be removed suitably by filtration, centrifugation, decantation, and other techniques. The solution may be filtered by passing through paper, glass fiber, or other membrane material, or a bed of a clarifying agent such as celite. Depending upon the equipment used and the concentration and temperature of the solution, the filtration apparatus may need to be preheated to avoid premature crystallization.

Removal of the solvent may be carried out suitably using techniques such as atmospheric evaporation or evaporation under vacuum, atmospheric distillation or distillation under vacuum, and the like.

Suitable techniques which may be used for solvent removal include spray drying, distillation using a rotational evaporator device such as a Buchi Rotavapor, freeze drying (lyophilization), spray drying and agitated thin film drying (“ATFD”).

Evaporation of the solvent may be conducted under a vacuum, such as below about 100 mm Hg, or below about 600 mm Hg, at temperatures such as about −20° C. to about 70° C. Any temperature and vacuum conditions may be used as long as there is no increase in the impurity levels of the product.

For example, spray drying, ATFD and evaporation by Buchi Rotavapor are more suitable for industrial scale production with batch sizes of about 100 g or about 1 Kg, or greater.

According to the present invention the obtained amorphous form from spray drying or a Buchi Rotavapor is quickly dissolved from pharmaceutical compositions.

The amorphous material obtained from step b) can be collected from the equipment using techniques such as by scraping, or by shaking the container, or using techniques specific to the particular apparatus, optionally under an inert gas atmosphere.

Optionally, drying of solid product may be carried out under suitable conditions to afford the desired lenalidomide in an amorphous form, substantially free of residual solvents.

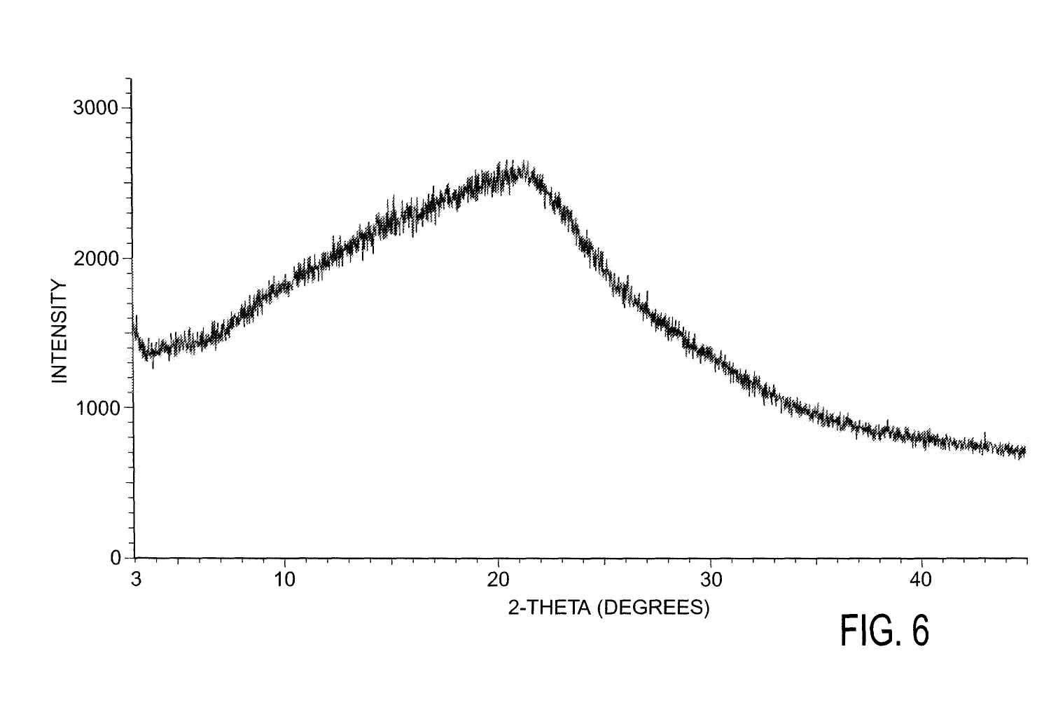

In an embodiment, there is provided a solid dispersion of lenalidomide and a pharmaceutically acceptable carrier, characterized by its XRD pattern substantially in accordance with FIG. 6.

In a further embodiment, there is provided a process for preparing amorphous lenalidomide, comprising removing solvent from a solution of lenalidomide and a pharmaceutically acceptable carrier.

In an embodiment, a process for preparing a solid dispersion containing amorphous lenalidomide comprises removing solvent from a solution of lenalidomide in combination with a pharmaceutically acceptable carrier.

A solution of lenalidomide may be provided by dissolving lenalidomide in a solvent or a mixture of solvents, or such a solution may be obtained directly from a reaction in which lenalidomide is formed. Any polymorphic form may be used in the preparation of solution, such as crystalline forms including solvates and hydrates.

Lenalidomide and the pharmaceutically acceptable carrier may be dissolved either in the same solvent or they may be dissolved in different solvents and then combined to form a mixture. In embodiments, the solid dispersion described herein includes lenalidomide and the carrier present in weight ratios ranging from about 5:95 to about 95:5. An example of a ratio is about 50:50.

Pharmaceutically acceptable carriers that may be used for the preparation of solid dispersions containing amorphous lenalidomide include, but are not limited to, pharmaceutical hydrophilic carriers such as polyvinylpyrrolidones (homopolymers of N-vinylpyrrolidone, called povidones), copolymers of N-vinylpyrrolidone, gums, cellulose derivatives (including hydroxypropyl methylcelluloses, HPMC), hydroxypropyl celluloses, mannitol and others), cyclodextrins, gelatins, hypromellose phthalates, sugars, polyhydric alcohols, polyethylene glycols, polyethylene oxides, polyoxyethylene derivatives, polyvinylalcohols, propylene glycol derivatives, and the like. The use of mixtures of more than one of the pharmaceutical carriers to provide desired release profiles or for the enhancement of stability is within the scope of this invention. Also, all viscosity grades, molecular weights, commercially available products, their copolymers, and mixtures are all within the scope of this invention without limitation.

Solvents which may be used for dissolving lenalidomide and pharmaceuticall acceptable carriers include, but are not limited to, water, organic solvents like C₁-C₄ alcohols, C₁-C₄ alkyl nitriles, C₃-C₅ alkyl amides, C₃-C₉ ketones, and mixtures thereof. Specific examples of solvents that may be utilized for the present invention include methanol, acetonitrile, N,N-dimethylformamide, N,N-dimethylacetamide, acetone, and mixtures thereof.

The dissolution temperatures can range from about 20° C. to about 100° C., depending on the solvent used for dissolution. Any other temperature is also acceptable as long as clear solutions are provided.

The quantity of solvent used for dissolution depends on the solvent and the dissolution temperature adopted. The concentration of lenalidomide in the solution may generally range from about 0.1 to about 10 g/ml in the solvent.

Optionally, the solution obtained above may be filtered to remove any undissolved particles before further processing. The undissolved particles may be removed suitably by filtration, centrifugation, decantation, and other techniques. The solution may be filtered by passing through paper, glass fiber, or other membrane material, or a clarifying agent such as celite. Depending upon the equipment used and the concentration and temperature of the solution, the filtration apparatus may need to be preheated to avoid premature crystallization.

Removal of the solvent may be carried out suitably using techniques such as atmospheric evaporation or evaporation under vacuum, atmospheric distillation or distillation under vacuum, and the like.

Suitable techniques which may be used for solvent removal include spray drying, distillation using a rotational evaporator device such as a Buchi Rotavapor, freeze drying (lyophilization), spray drying and agitated thin film drying (“ATFD”).

Evaporation of the solvent may be conducted under a vacuum, such as below about 100 mm Hg, or below about 600 mm Hg, at temperatures such as about −20° C. to about 70° C. Any temperature and vacuum conditions may be used as long as there is no increase in the impurity levels of the product.

For example, spray drying, ATFD and evaporation by Buchi Rotavapor are more suitable for industrial scale production with a batch size of about 100 g or about 1 Kg, or greater.

According to the present invention the obtained amorphous form by spray drying or Buchi Rotavapor is quickly dissolved from pharmaceutical compositions.

The amorphous material obtained can be collected from the equipment using techniques such as by scraping, or by shaking the container, or using techniques specific to the particular apparatus optionally under nitrogen atmosphere.

Optionally, drying of solid product may be carried out under suitable conditions to afford the desired solid dispersion of lenalidomide in an amorphous form, substantially free of residual solvents.

In embodiments, a solid dispersion of lenalidomide contains residual solvents greater than about 1% and less than about 10% with respect to the weight of the solid dispersion. In a particular embodiment, a solid dispersion has a residual solvent content less than about 2% by weight. In another embodiment, a solid dispersion has a residual solvent content ranging from about 4% to about 7%, by weight.

Drying may be carried out until the residual solvent content reduces to a desired amount, such as an amount that is within the limits given by the International Conference on Harmonisation of Technical Requirements for Registration of Pharmaceuticals for Human Use (“ICH”) guidelines. The guideline solvent level depends on the type of solvent but is not more than about 5000 ppm, or about 4000 ppm, or about 3000 ppm.

The drying may be carried out at reduced pressures, such as below about 650 mm Hg, or below about 50 mm Hg, at temperatures such as about 35° C. to about 70° C. The drying may be carried out for any desired time period that achieves the desired result, such as times about 1 to 20 hours, or longer. Drying may also be carried out for shorter or longer periods of time depending on the product specifications.

Drying may be suitably carried out in equipment such as a tray dryer, vacuum oven, air oven, or using a fluidized bed drier, spin flash dryer, flash dryer, and the like.

In a further aspect, there are provided processes for preparing amorphous lenalidomide, an embodiment comprising milling a lenalidomide crystalline material to afford the amorphous form of lenalidomide. The specifics of an embodiment of a process are provided in Example 9.

Lenalidomide and its impurities may be analyzed using HPLC, for example using the following set of conditions:

Instrument: Waters 2695 separation module with 2996 PDA detector.

Column: 250×4.6 mm, 5 μm (Waters Xterra R-18).

Buffer: 1.36 g of potassium dihydrogen orthophosphate anhydrous is dissolved in 100 mL of milli-Q water, pH of the solution is adjusted to 3.5±0.05 using dilute phosphoric acid, and the solution is filtered through a 0.45 μm membrane filter.

Mobile Phase A: Buffer.

Mobile Phase B: Filtered and degassed mixture of methanol and acetonitrile in the ratio of 90:10 by volume.

Flow rate: 1.0 mL/minute.

Wavelength of detection: 210 nm.

Column temperature: Ambient.

Injection volume: 10 μL.

Run time: 60 minutes.

Diluent: Mobile phase A and mobile phase B (1:1 by volume).

Gradient program:

| Time (minutes) | Mobile Phase A | Mobile Phase B |
|---|---|---|
| 0 | 90 | 10 |
| 15 | 90 | 10 |
| 40 | 45 | 55 |
| 52 | 45 | 55 |
| 53 | 90 | 10 |
| 60 | 90 | 10 |

A sample is prepared for analysis by placing an accurately weighed amount that contains about 50 mg of lenalidomide into a 50 mL volumetric flask, dissolving the lenalidomide content in diluent solution, and diluting to volume with the diluent. A portion can be filtered before injection into the chromatograph.

The same method may also be utilized for analyzing the purity of lenalidomide salts including a methanesulfonate salt of lenalidomide.

In embodiments, lenalidomide obtained by the processes of the invention contains less than about 0.1% by weight, as determined using HPLC, of any of the individual impurities listed in Table 1.

TABLE 1

| Impurity | Structure | RRT* |
|---|---|---|
| Impurity A | 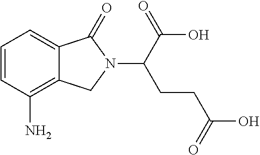 | 0.66 |
| Impurity B | 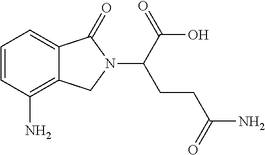 | 0.8 |
| Impurity C | Undetermined | 1.74 |
| Impurity D | 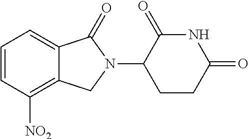 | 1.98 |

*Relative retention time, lenalidomide = 1.

In an embodiment, the present invention provides lenalidomide having a purity at least about 99.8% by weight, as determined using HPLC.

In an embodiment, the present invention provides lenalidomide having a purity at least about 99.8% by weight, and containing less than about 0.1% by weight of Impurity C, as determined using HPLC.

Certain specific aspects and embodiments of the invention will be explained in more detail with reference to the following examples, which are provided solely for purposes of illustration and should not be construed as limiting the scope of the invention in any manner. In the examples, percentages are by weight unless the context clearly indicates otherwise.

### Example 1

### PREPARATION OF 3-(4-NITRO-1-OXO-1,3-DIHYDROISOINDOL-2-YL)-PIPERIDINE-2,6-DIONE (FORMULA II)

Methyl 2-bromomethyl-3-nitrobenzoate (2.2 Kg) is dissolved in acetonitrile (22 L) and placed into a glass container. α-Amino glutarimide hydrochloride (1.32 Kg) is added to the solution at 28° C. and stirred for 10 minutes. Triethylamine (0.56 L) is added under a nitrogen atmosphere and the mixture heated to a temperature of 55° C., and then the mixture stirred for 2 hours. The triethylamine addition, heating, and stirring are repeated 3 times and then the reaction mixture is stirred for 18 hours at 50° C. After completion of the reaction, the reaction mixture is cooled to 28° C. Demineralized water (7 L) is charged to the reaction mixture and then stirred for 2 hours at 28° C. The reaction mixture is filtered and the solid is dried at 45° C. under a vacuum of 600 mm Hg for 8-9 hours to afford 2 Kg of the title compound, with a purity by HPLC of 99.07%.

### Example 2

### PREPARATION OF A METHANESULFONATE SALT OF 3-(4-AMINO-1-OXO-1,3-DIHYDROISOINDOL-2-YL)-PIPERIDINE-2,6-DIONE

3-(4-nitro-1-oxo-1,3-dihydroisoindol-2-yl)piperidine-2,6-dione (10 g), methanol (300 mL), 10% palladium on carbon (0.3 g) and methanesulfonic acid (4.5 mL; d: 1.48) are charged into a conical flask and then transferred into an autoclave. Hydrogen gas (90 psi, 6.3 Kg/cm²) is applied to the suspension at 30° C. and stirred for 3-4 hours. The reaction mixture is filtered through a celite bed and the bed is washed with methanol (20 mL). The obtained filtrate is concentrated until the reaction mass becomes about 100 mL and stirred for 20 minutes. The reaction mass is filtered and dried the solid dried for 4 hours at 50° C., to give 8 g of a methanesulfonate salt of lenalidomide.

Purity by HPLC 99.87%.

Impurity A 0.01%, Impurity B 0.01%, Impurity C 0.04%, Impurity D not detected.

XRD pattern substantially in accordance with FIG. 1.

DSC curve substantially in accordance with FIG. 2.

TGA weight loss 0.77% w/w; curve substantially in accordance with FIG. 3.

### Example 3

### Preparation of Lenalidomide

A methanesulfonate salt of lenalidomide (1.0 g) and isopropanol (6 mL) are charged into a round bottom flask and stirred. Triethylamine (0.4 mL) is added to the mixture and stirred for 50 minutes. Isopropanol (2 mL) is added to the mixture with stirring for 30 minutes. The reaction mass is filtered, washed with isopropanol (2 mL) and the solid dried at 48° C. under a vacuum of 600 mm Hg for a period of 3 hours, to afford 680 mg of lenalidomide (yield, 93%).

Purity by HPLC 99.86%.

XRD pattern is substantially in accordance with FIG. 7.

### Example 4

### In Situ Preparation of Lenalidomide

3-(4-nitro-1-oxo-1,3-dihydroisoindol-2-yl)-piperidine-2,6-dione (10 g), water (300 mL), 10% palladium on carbon (1 g, 50% wet) and methanesulfonic acid (5 mL) are charged into a conical flask and then transferred into an autoclave. Dry hydrogen gas pressure (30-35 psi, 2.1-2.5 Kg/cm²) is applied to the suspension at 30° C. for 2-3 hours and then the reaction mixture is filtered through a celite bed. The obtained filtrate is neutralized with 7% sodium bicarbonate solution (90 mL) and stirred for 1 hour. The solid obtained is filtered and dried at 45° C. under a vacuum of 600 mm Hg for 2 hours, to yield 5.16 g of crystalline Form B of Lenalidomide.

Purity 99.74% by HPLC.

XRD pattern substantially in accordance with FIG. 8.

Impurities by HPLC:

3-Amino-piperidine-2,6-dione hydrochloride (Impurity A) 0.01%.

3-(4-Nitro-1-oxo-1,3-dihydro-isoindol-2-yl)-piperidine-2,6-dione (Impurity B) 0.02%.

Highest unidentified impurity 0.04%.

### Example 5

### Preparation of Crystalline Form B of Lenalidomide

Lenalidomide (3 g) obtained from Example 3 is suspended in water (30 mL) and stirred for 6 hours at 70-75° C. The suspension is cooled to 60° C. and then filtered. The resultant solid is dried at 45° C. under reduced pressure for 4-5 hours to afford 2.68 g of product. Purity 99.89%.

Impurities:

3-Amino-piperidine-2,6-dione hydrochloride (Impurity A) not detected.

3-(4-Nitro-1-oxo-1,3-dihydro-isoindol-2-yl)-piperidine-2,6-dione (Impurity B) 0.009%.

Highest impurity 0.06%.

### Example 6

### Preparation of Lenalidomide

3-(4-nitro-1-oxo-1,3-dihydroisoindol-2-yl)-piperidine-2,6-dione (10 g), methanol (200 mL), 10% palladium on carbon (0.3 g, 50% wet) and methanesulfonic acid (2.24 mL) are charged into a conical flask and then transferred into an autoclave. Dry hydrogen gas pressure (30-35 psi, 2.1-2.5 Kg/cm²) is applied to the suspension at 30° C. for 4 hours and then the reaction mixture is filtered through a celite bed and washed with methanol (100 mL). The obtained filtrate is concentrated to a 100 mL volume at 40-45° C. and then neutralized with 7% sodium bicarbonate solution (45 mL) followed by stirring the suspension for 1-2 hours at 25-35° C. The solid produced is filtered and dried at 45° C. under a vacuum of 600 mm Hg for 3-4 hours, to yield 7 g of the crystalline Form B of lenalidomide (yield, 78%).

Purity 99.61%.

Impurities:

3-Amino-piperidine-2,6-dione hydrochloride (Impurity A) not detected.

3-(4-Nitro-1-oxo-1,3-dihydroisoindol-2-yl)piperidine-2,6-dione (Impurity B) 0.04%.

Highest impurity 0.17%.

### Example 7

### Preparation of Lenalidomide

### A) Preparation of 3-(4-nitro-1-oxo-1,3-dihydroisoindol-2-yl)-piperidine-2,6-dione of Formula II

Methyl 2-bromomethyl-3-nitrobenzoate (100 g) is dissolved in N-methylpyrrolidone (1 L) at a temperature of 25-30° C. α-Aminoglutarimide hydrochloride (60 g) and triethylamine (25.4 mL) are charged to the solution and stirred for 2 hours. The triethylamine addition and stirring are repeated 3 times and then the reaction mixture is stirred for a period of 1 to 2 hours at a temperature of 25-30° C. Demineralized water (300 mL) is added to the reaction mixture and then stirred for 1 hour. The suspension is filtered and the solid dried at 50° C. under a vacuum of 600 mm Hg for a period of 8-9 hours, to afford 84 g of the compound of Formula II.

### B) Preparation of lenalidomide of Formula I

3-(4-nitro-1-oxo-1,3-dihydroisoindol-2-yl)piperidine-2,6-dione (10 g), water (150 mL), 10% palladium on carbon (0.5 g, 50% wet) and methanesulfonic acid (5.6 mL; d: 1.48) are charged into a conical flask and then transferred into an autoclave. Hydrogen gas (90 psi, 6.3 Kg/cm²) is applied to the suspension at 30° C. and stirred for 3 hours. The reaction mixture is filtered through a celite bed and the bed washed with water (50 mL). The obtained filtrate is neutralized with 7% sodium bicarbonate solution (100 mL) and stirred for 1 hour. The reaction suspension obtained is filtered and the solid dried at 50° C. under a vacuum of 600 mm Hg for 5-6 hours, to yield 7.2 g of lenalidomide.

Purity by HPLC 99.93%.

### Example 8

### Amorphous Lenalidomide

Lenalidomide (2 g) is added to methanol (25 mL) and dimethylformamide (25 mL) is added at 34° C. The mixture is stirred at the same temperature to produce a clear solution. The clear solution is heated to 60° C. for 2 minutes. The resultant solution is evaporated completely using a Buchi Model No: B290 spray dryer and the following conditions:

Aspirator: 70%.

Feed rate: 20%.

Inlet Temperature: 120° C.

N₂ pressure: 5.0 kg/cm².

The obtained material is collected under a nitrogen atmosphere as an amorphous solid and packaged in a polyethylene bag. Yield: 0.9 g (45%).

The material remains amorphous for five days at 0-5° C.

### Example 9

### Amorphous Lenalidomide

Lenalidomide (1 g) is placed into a ball mill with stainless steel 316 balls, and operated with the conditions:

Temperature: 34° C.

Time: 2 hours.

Speed: 300 rpm.

Reverse rotation every 10 minutes.

The obtained material is collected under a nitrogen atmosphere as an amorphous solid and packaged in a polyethylene bag. Yield: 0.9 g (about 90%).

### Example 10

### Solid Dispersion of Lenalidomide

Lenalidomide (15 g) is dissolved in N,N-dimethylformamide (210 mL) at a temperature of 70° C. and povidone K-30 (15 g) is dissolved in methanol (150 mL). The solutions are combined, filtered, concentrated completely at about 110-120° C. and dried for 2-3 hours at 100° C. to obtain 20.5 g of the dispersion.

The material remains amorphous for 60 days at ambient temperature (25-30° C.).

The obtained material has an XRD pattern that is substantially in accordance with FIG. 6.

### Example 11

### Solid Dispersion of Lenalidomide

Lenalidomide (15 g) is dissolved in N,N-dimethylformamide (210 mL) at a temperature of 70° C. and povidone K-30 (15 g) is dissolved in methanol (150 mL) at 60° C. The solutions are combined and filtered. The resultant solution is evaporated completely using a spray dryer with the following parameters:

Aspirator: 70%.

Feed rate: 20%.

Inlet temperature: 160° C.

N₂ pressure: 6.0 kg/cm².

The material obtained by spray drying was amorphous. Yield: 14.1 g (47%).

The obtained material (8 g) is micronized with a gas jet mill under a nitrogen atmosphere for 10 minutes, and is packaged together with a silica gel desiccant in a polyethylene bag, placed in a sealed triple laminated outer bag.

The material remains amorphous for 3 months at room temperature and at 2-8° C.

### Example 12

### Preparation of Lenalidomide

A methanesulfonate salt of lenalidomide (1.0 g) and methanol (2 mL) are charged into a round bottom flask and stirred at room temperature. Triethylamine (0.4 mL) and methyl t-butyl ether (5 mL) are added to the mixture and stirred for 1 hour. The mass is filtered, washed with a mixture of methanol and methyl t-butyl ether (1:1 by volume, 2 mL) and the solid obtained is dried at 48° C. under a vacuum of 600 mm Hg for a period of 4 hours, to afford 650 mg of lenalidomide

(yield, 89%).

Purity by HPLC 99.80%.

Impurity A not detected; Impurity B 0.06%; Impurity C 0.01%; Impurity D 0.02%

XRD pattern is substantially in accordance with FIG. 7.

1. Amorphous lenalidomide.

2. The amorphous lenalidomide of claim 1, having an X-ray powder diffraction pattern substantially in accordance with the pattern of any of FIG. 4, 5, or 6.

3. A solid dispersion comprising lenalidomide and at least one pharmaceutically acceptable carrier.

4. The solid dispersion of claim 3, wherein lenalidomide is in amorphous form.

5. The solid dispersion of claim 3, wherein a pharmaceutically acceptable carrier comprises one or more of a polyvinylpyrrolidone, a cellulose derivative, a polyhydric alcohol, a polyethylene glycol, a polyethylene oxide, a polyoxyethylene derivative, a polyvinyl alcohol, and a propylene glycol derivative.

6. A process for preparing amorphous lenalidomide or a solid dispersion containing lenalidomide, comprising removing solvent from a solution comprising lenalidomide and optionally a pharmaceutically acceptable carrier.

7. The process of claim 6, wherein a solvent comprises one or more of an alcohol having 1-4 carbon atoms, an alkyl nitrile having 1-4 carbon atoms, an alkyl amide having 3-5 carbon atoms, and a ketone having 3-9 carbon atoms.

8. The process of claim 6, wherein removing solvent comprises at least one of vacuum distillation, spray drying, and freeze drying.

9. The solid dispersion of claim 3, wherein a pharmaceutically acceptable carrier comprises povidone K-30.

10. Lenalidomide having a purity of at least about 99% by weight.

11. A process for preparing substantially pure lenalidomide, comprising:
i) reacting a methyl 2-halomethyl-3-nitrobenzoate of Formula III, where X is a halogen, 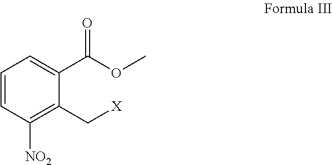
with α-aminoglutarimide hydrochloride of Formula IV, 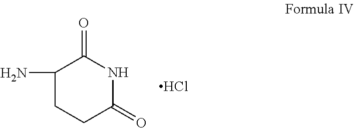
using triethylamine in the presence of a solvent, to afford 3-(4-nitro-1-oxo-1,3 dihydro-isoindol-2-yl)-piperidine-2,6-dione of Formula II; and 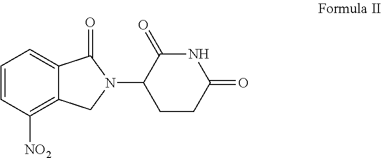
ii) hydrogenating 3-(4-nitro-1-oxo-1,3 dihydroisoindol-2-yl)-piperidine-2,6-dione of Formula II using a hydrogenation catalyst in a solvent and in the presence of an acid, to provide lenalidomide.

12. The process of claim 11, wherein a solvent comprises N-methylpyrrolidone or acetonitrile.

13. The process of claim 11, wherein a hydrogenation catalyst comprises palladium on carbon.

14. The process of claim 11, wherein and acid comprises one or more of: an organic acid comprising methanesulfonic acid, an arylsulfonic acid, formic acid, acetic acid, trifluoroacetic acid, or a salt thereof; or an inorganic acid comprising hydrochloric acid, sulfuric acid, or phosphoric acid.

15. A process for the preparation of a methanesulfonate acid salt of lenalidomide comprising: hydrogenating the compound 3-(4-nitro-1-oxo-1,3-dihydroisoindol-2-yl)-piperidine-2,6-dione of Formula II, 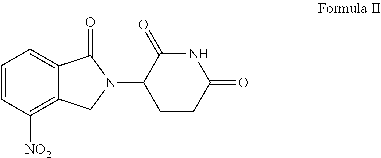
using a hydrogenation catalyst in the presence of a solvent and methanesulfonic acid.

16. The process of claim 15, wherein a hydrogenation catalyst comprises palladium on carbon having a palladium content from about 1 to about 30% by weight.

17. The process of claim 15, wherein a hydrogenation catalyst comprises palladium on carbon having a palladium content about 10% by weight.

18. A crystalline methanesulfonate salt of lenalidomide, characterized by an X-ray powder diffraction pattern having peak locations substantially in accordance with FIG. 1.
